(19) United States
(12) Patent Application Publication
(10) Pub. No.: US 2020/0074162 A1
(43) Pub. Date: Mar. 5, 2020

(54) BIOKINETIC ANALYSIS SYSTEM WITH INSTRUMENTED WALKWAY AND 3D VIDEO CAMERA

(71) Applicant: Douglas D Haas, Sparta, NJ (US)

(72) Inventor: Douglas D Haas, Sparta, NJ (US)

(21) Appl. No.: 16/552,869

(22) Filed: Aug. 27, 2019

Related U.S. Application Data
(60) Provisional application No. 62/723,486, filed on Aug. 28, 2018.

(51) Int. Cl. G06K 9/00; A61B 5/11; G06T 7/20; H04N 21/4223

(52) U.S. Cl. CPC G06K 9/00348; A61B 5/112; A61B 5/1116; G06K 9/6232; H04N 21/4223; G06K 9/00771; A61B 5/1123; G06T 7/20

(57) ABSTRACT

A biokinetic analysis system is used to analyze the movement of a human subject, and coordinate the foot motion with the body motion. The subject walks upon an instrumented walkway, and sensors detect pressure of the subject feet. A 3D camera is aligned with a target on the walkway, and captures movement of the subject body toward the camera. The camera lenses are spaced apart vertically. Data from the walkway and data from the camera are captured simultaneously, and sent to a computer to analyze and store. Electrical power is supplied alternately by an electrical storage battery, or a photovoltaic module, or the grid. A unitary interface module electrically connects conductors to the battery, the photovoltaic module, the walkway, the camera, and the computer. The unitary interface module also digitally connects the walkway, the camera, and the computer, conveying power and data simultaneously. A transport case is provided.

### INCORPORATION BY REFERENCE

U.S. Pat. No. 5,952,585, issued Sep. 14, 1999, entitled “Portable Pressure Sensing Apparatus For Measuring Dynamic Gait Analysis And Method Of Manufacture,” is incorporated in its entirety for the teachings therein.

U.S. Provisional patent application Ser. No. 62/723,486, filed on 28 Aug. 2018, entitled “Biokinetic Analysis System With Instrumented Walkway And 3D Video Camera,” is incorporated in its entirety for the teachings therein. The priority filing date of 28 Aug. 2018 is hereby claimed.

### TECHNICAL FIELD

The presently disclosed technologies are directed to an apparatus and method for analysis of human biokinetics by walking a subject upon an instrumented walkway and simultaneously videographing the subject with a 3d video camera, and integrating the data digitally with a computer.

### BACKGROUND

Physicians and research scientists need to know how the body moves normally, and compare that with impaired movement, in order to diagnose and treat the impairment. The ability to record how the body is moving, analyze the movement, and prepare a report is necessary in studying the biokinetics of a subject. The portions of the body that are impaired, as well as the degree of impairment, must be understood in order to plan a treatment regimen. The nature of the condition, as well as any changes over time, such as the degree of improvement or worsening of the condition must be determined. This information allows the formulation of therapy, and is useful in Orthopedics, Neurology, Podiatry, Geriatrics, and other specialties. In the field of Geriatrics for example, it is advantageous to determine the likelihood that a subject will fall down, and how that probability changes over time.

For many years there have been a number of systems that record body movement using a large number of 3D cameras in a dedicated laboratory. Recently developed systems utilize multiple sensors worn by the subject. The sensors identify specific parts of the body being recorded for analysis. One problem with this system is that the sensors must be placed in exactly the same place on the subject every time the subject is tested. Another problem is accounting for the signal drift that occurs by the nature of the sensors. In the case of the multi-camera video system, careful calibration of the multiple cameras is required. This system further requires well trained technicians. An additional problem is that the system does not provide accurate timing of the placing down and lifting up of the foot. Nor does the system provide any pressure information for the foot transition during weight transfer from heel to toe.

Body and gait analysis systems are known, and have taken a variety of configurations in the past. Some examples are shown in the following prior art.

Ecker, U.S. Pat. No. 9,311,540; requires cameras in each patient room of a facility, and constant monitoring by skilled observers looking for an overall body pattern; alerts an actual fall, does not assess risk of falling.

Yang, US Pub No. 2007/0104351; uses a camera to track the subject, but concerns image processing only, not gait analysis; does not assess risk of falling.

Cutler, U.S. Pat. No. 7,330,566; a camera system attempts to identify a subject based upon gait; does not assess risk of falling.

Cuddihy, U.S. Pat. No. 8,508,372; additional reference devices are required; alerts an actual fall, does not assess risk of falling.

Barralon, European Patent Application No. EP 3 017 761; attaches the camera to the subject, requires additional sensors, and an infrared light source; subject stands on a pressure plate; does not assess risk of falling.

Steele, U.S. Pat. No. 9,836,118; uses force sensors placed on subject; does not assess risk of falling.

Willmann, International Pub. No. WO 2008/129442; uses sensors placed on subject; tracks progress during therapy for stroke patients; does not assess risk of falling.

Rehg, U.S. Pat. No. 6,269,172; method for analyzing 3D images; requires articulated physical model; does not assess risk of falling.

Oba, International Pub. No. WO 2004/103176; uses sensors placed on subject; no camera is used; does not assess risk of falling.

Laett, U.S. Pat. No. 9,501,919; used to monitor patients position in a designated area; does not assess risk of falling.

Guimond, US Pub. No. 2003/0181830; uses sensors placed on subject; does not assess risk of falling.

Krebs, US Pub. No. 2002/0028003; subject identification by gait; does not assess risk of falling.

Goebel, International Pub. No. WO 2016/081994; camera is mounted on a walker; does not assess risk of falling.

Fujieda, US Pub. No. 2010/0232683; multiple cameras for subject identification; does not assess risk of falling.

Even-Zohar, U.S. Pat. No. 6,774,885; subject standing on moving platform; uses sensors placed on subject; does not assess risk of falling.

Stone, U.S. Pat. No. 9,597,016; requires continuous monitoring by a trained observer in a medical facility; does not use instrumented walkway data integrated with camera data.

Reinhold, US Publication No. 2012/0253201; requires infrared light source and multiple cameras from side view; does not use instrumented walkway data integrated with camera data.

Daniel, US Publication No. 2003/0228033; requires eleven tests with monitoring and analysis by a trained observer in a medical facility; does not use instrumented walkway data integrated with camera data.

Bunn, International Pub. No. WO 2009/111886; a camera and algorithm determines illness of subject; requires database of similar subjects for reference; requires analysis by a trained observer in a medical facility; does not use instrumented walkway data integrated with camera data.

Annegarn, International Pub. No. WO 2018/069262; uses sensors placed on subject to identify fall risk.

Park, U.S. Pat. No. 7,239,718; system records motion using high speed 3D for the production of movies; does not assess risk of falling.

Hardigan, US Pub. No. 2011/0082672; system for analyzing statistical data related to various diseases; no camera or walkway is employed; does not specify data source.

Different systems are available for analysis of human gait by walking a subject upon an instrumented walkway. An array of sensors disposed on the walkway detect foot pressure. Data is sent to a computer for analysis. The portable electronic pressure walkway system enabled gait analysis in a physician's office. Analysis is carried out conveniently where the subject/patient is located. Highly trained technicians are not needed. Nor is specialized computer expertise. An example of this temporal/spatial walkway system is found in U.S. Pat. No. 5,952,585, and is manufactured by CIR Systems. The portable walkway system works very well for gait analysis, but does not collect data on the movement of the upper body or limbs.

The portable electronic pressure walkway system must be connected to an electrical power grid. In a physician's office, this is not a problem. In a rural setting, or in a second-world country, reliable grid power can be an issue. The above-described systems currently available must be powered reliably. Where reliable grid power is not available, batteries are an option, but they must be recharged regularly. The charging means must be portable and integrated with the system. The charging means must also be easily operated by personnel with minimal training.

In a rural setting, or in a second-world country, the system must be self-contained and portable for easy transport and set-up.

Accordingly, there is a need to provide a biokinetic analysis system capable of analyzing gait, and of analyzing movement of the body simultaneously, and of recording the data in a computer, wherein data capture of the feet and data capture of the body is synchronized in real time, thereby avoiding the problems associated with the prior art.

There is a further need to provide a biokinetic analysis system of the type described, and that requires only one 3D camera and no sensors attached to the subject.

There is a yet further need to provide a biokinetic analysis system of the type described, and that can be set up quickly anywhere, and operated easily by a clinician with minimal training, and provide immediate results readily interpreted.

There is a still further need to provide a biokinetic analysis system of the type described, and which is self-contained, is portable, can be operated on grid power or powered by batteries, and has charging means not dependent upon grid power.

There is an additional need to provide a biokinetic analysis system of the type described, and that is cost-effective and robust for reliable service life.

### SUMMARY

In one aspect, a biokinetic analysis system is used in connection with a human subject having a subject body and subject feet. The biokinetic analysis system comprises an instrumented walkway extending from a first end to an opposite second end, and adapted for lying upon a floor. The walkway has an array of sensors disposed on the walkway so as to detect pressure of the subject feet walking upon the walkway as subject feet data. The walkway is adapted to convey the subject feet data digitally.

A 3D camera is disposed adjacent the walkway. The camera has two lenses aimed toward the walkway. The camera is adapted to optically capture movement of the subject body on the walkway as subject body data. The camera is adapted to convey the subject body data digitally.

A computer is operatively digitally connected to the walkway and to the camera. The computer is adapted to receive the subject feet data from the walkway and the subject body data from the camera simultaneously. The computer is programmed to analyze and store the data.

A walkway power means supplies electrical power, and is operatively electrically connected to the walkway. A camera power means supplies electrical power, and is operatively electrically connected to the camera. A computer power means supplies electrical power, and is operatively electrically connected to the computer.

A data means conveys data, and is operatively digitally connected to the walkway, the camera, and the computer. Thus, the subject will walk from the walkway first end toward the walkway second end. The data from the walkway and the data from the camera are captured simultaneously.

In another aspect, a biokinetic analysis system is used in connection with a human subject having a subject body and subject feet. The biokinetic analysis system comprises an instrumented walkway extending from a first end to an opposite second end, and adapted for lying upon a floor. The walkway has a target disposed on the walkway adjacent the second end of the walkway. The walkway has an array of sensors disposed on the walkway so as to detect pressure of the subject feet walking upon the walkway as subject feet data. The walkway is adapted to convey the subject feet data digitally.

A 3D camera is disposed above the target adjacent the second end of the walkway and spaced apart from the array of sensors. The camera has two lenses aimed longitudinally to the walkway and toward the first end of the walkway. The camera is adapted to optically capture movement toward the camera of the subject body on the walkway as subject body data. The camera is adapted to convey the subject body data digitally.

A computer is operatively digitally connected to the walkway and to the camera. The computer is adapted to receive the subject feet data from the walkway and the subject body data from the camera simultaneously. The computer is programmed to analyze and store the data.

A system power means supplies electrical power, and is operatively electrically connected to the walkway, the camera, and the computer. The system power means is selected from one of the following: at least one electrical storage battery; a photovoltaic module adapted for receiving solar light and converting the light into electrical power; and a power supply operatively electrically connected to utility grid power.

A data means conveys data, and is operatively digitally connected to the walkway, the camera, and the computer. Thus, the subject will walk from the walkway first end toward the walkway second end. The data from the walkway and the data from the camera are captured simultaneously.

In yet another aspect, a biokinetic analysis system is used in connection with a human subject having a subject body and subject feet. The biokinetic analysis system comprises an instrumented walkway extending from a first end to an opposite second end, and adapted for lying upon a floor. The walkway has a target disposed on the walkway adjacent the second end of the walkway. The walkway has an array of sensors disposed on the walkway so as to detect pressure of the subject feet walking upon the walkway as subject feet data. The walkway is adapted to convey the subject feet data digitally. The walkway has a centerline walkway axis extending between the first and second ends and centered transversely on the array of sensors.

A 3D camera is disposed above the target adjacent the second end of the walkway and spaced apart from the array of sensors. The camera has two lenses aimed longitudinally to the walkway and toward the first end of the walkway. The lenses each have a centerline lens axis and are disposed with the lenses spaced apart one above the other. Each lens axis and the walkway axis lie generally within a plane that is generally vertical. The camera will optically capture movement toward the camera of the subject body on the walkway as subject body data. The camera will also convey the subject body data digitally.

A computer is operatively digitally connected to the walkway and to the camera. The computer receives the subject feet data from the walkway and the subject body data from the camera simultaneously. The computer is programmed to analyze and store the data. Thus, the subject will walk from the walkway first end toward the walkway second end, and the data from the walkway and the data from the camera will be captured simultaneously.

A unitary interface module includes a system power means for operatively electrically connecting the walkway, the camera, and the computer. The unitary interface module also includes a data means operatively digitally connecting the walkway, the camera, and the computer. In this manner, both power and data are conveyed simultaneously, and along the same conductors.

A path image is generated by software and displayed on the computer. A walkway image is captured by the camera and displayed on the computer. In the event that the camera is not properly aligned with the walkway, the walkway image and the path image will not coincide on screen. The walkway image and the path image will coincide when the camera is aligned with the walkway.

These and other aspects, objectives, features, and advantages of the disclosed technologies will become apparent from the following detailed description of illustrative embodiments thereof, which is to be read in connection with the accompanying drawings.

### BRIEF DESCRIPTION OF THE DRAWINGS

FIG. 1 is a side elevational view of an exemplary biokinetic analysis system constructed in accordance with the invention.

FIG. 2 is a top plan diagrammatic view of the biokinetic analysis system of FIG. 1, showing the components connected.

FIG. 3 is a front perspective view of a camera, a camera support, a camera mount, a camera support base, feet, and pockets, for use with the biokinetic analysis system of FIG. 1.

FIG. 4 is a front elevational computer graphical representation of a subject under analysis, showing subject body data, for use with the biokinetic analysis system of FIG. 1.

FIG. 5 is a front elevational view of the computer of the biokinetic analysis system of FIG. 1, showing the path and the walkway out of alignment.

FIG. 6 is a front elevational view of the computer of the biokinetic analysis system of FIG. 1, showing the path and the walkway in proper alignment.

FIG. 7 is a top plan diagrammatic view of another biokinetic analysis system constructed in accordance with the invention, and showing the components connected.

FIG. 8 is a top plan view of the biokinetic analysis system of FIG. 7, showing the components packed into a transport case.

FIG. 9 is an enlarged top plan detail view of the biokinetic analysis system of FIG. 7, slowing details of the batteries and power cables.

FIG. 10 is a top perspective detail view of the biokinetic analysis system of FIG. 7, slowing the batteries and power cables installed into the transport case.

FIG. 11 is a top isometric view of a battery for use in the biokinetic analysis system of FIG. 7.

FIG. 12 is a top isometric view of a battery pack and meter for use with the biokinetic analysis system of FIG. 7.

FIG. 13 is a top perspective view of the biokinetic analysis system of FIG. 7, showing the components packed into the transport case.

FIG. 14 is a top perspective view of the transport case.

FIG. 15 is a top perspective view of the solar photovoltaic module.

FIG. 16 is a side elevational view of yet another biokinetic analysis system constructed in accordance with the invention.

FIG. 17 is a top plan diagrammatic view of the biokinetic analysis system of FIG. 16, showing the components connected.

FIG. 18 is a front perspective view of a camera and a camera support for use with the biokinetic analysis system of FIG. 16.

It should be noted that the drawings herein are not to scale.

### DETAILED DESCRIPTION

As used herein, “gait analysis” refers to the study of walking of a human subject.

As used herein, “biokinetics” refers to the body movement of the subject.

As used herein, “3D camera” refers to a camera having two lenses spaced apart, each lens being optically connected to an optical sensor, and supplying three dimensional optical data in a digital format.

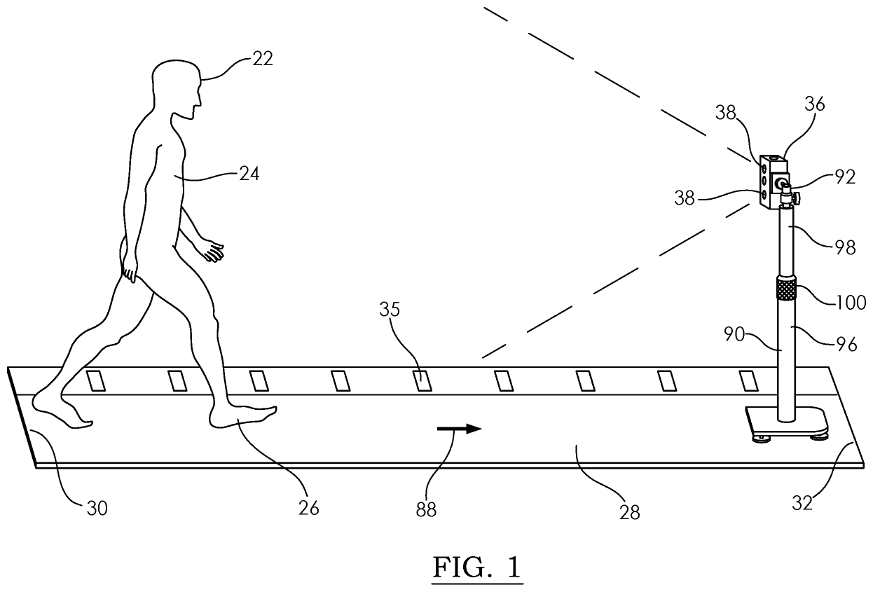

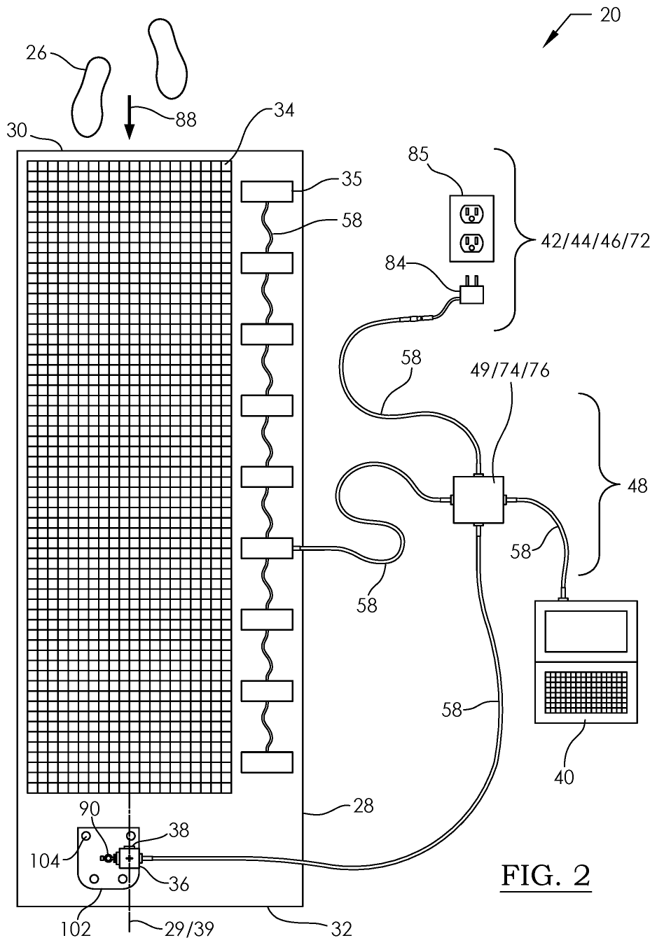

Describing now in further detail these exemplary embodiments with reference to the Figures as described above, the biokinetic analysis system 20 is used in connection with a human subject 22 having a subject body 24 and subject feet 26. The biokinetic analysis system 20 comprises an instrumented walkway 28 extending from a first end 30 to an opposite second end 32, as shown in FIGS. 1 and 2. The walkway 28 is flexible so that it can be rolled up for storage, and unrolled for use upon a floor. The walkway has a target 86 disposed on the walkway adjacent the second end of the walkway. The walkway 28 has an array of sensors 34 disposed on the walkway 28 so as to detect pressure of the subject feet 26 walking upon the walkway 28. The pressure is collected as subject feet data, and the walkway 28 will convey the subject feet data digitally. Circuit boards 35 gather data from the array of sensors 34. The circuit boards 35 are connected together and convey data through connector cables 58. The walkway has a centerline walkway axis 29 extending between the first 30 and second 32 ends and centered transversely on the array of sensors 34.

A 3D camera 36 is disposed above the target 86 adjacent the second end 32 of the walkway 28 and spaced apart from the array of sensors 34. The camera 36 has two lenses 38 aimed longitudinally to the walkway 28 and toward the first end 30 of the walkway 28, as shown in FIGS. 1 and 2. The lenses 38 each have a centerline lens axis 39 and are disposed with the lenses spaced apart one above the other. Each lens axis 39 and the walkway axis 29 lie generally within a plane that is generally vertical. The camera 36 will optically capture movement of the subject body 24 on the walkway 28 in the direction 88, toward the camera, as subject body data. The camera 36 will convey the subject body data digitally. The 3D camera 36 of the preferred embodiment captures images in either the visible or the infrared spectrum. Any wavelength of light compatible with the 3D camera will operate with the invention.

In FIG. 2, a computer 40 is operatively digitally connected to the walkway 28 and to the camera 36. The computer 40 receives the subject feet data from the walkway 28 and the subject body data from the camera 36 simultaneously. The computer 40 is programmed to analyze and store the data. Thus, the subject 22 will walk from the walkway first end 30 toward the walkway second end 32, and the data from the walkway 28 and the data from the camera 36 will be captured simultaneously.

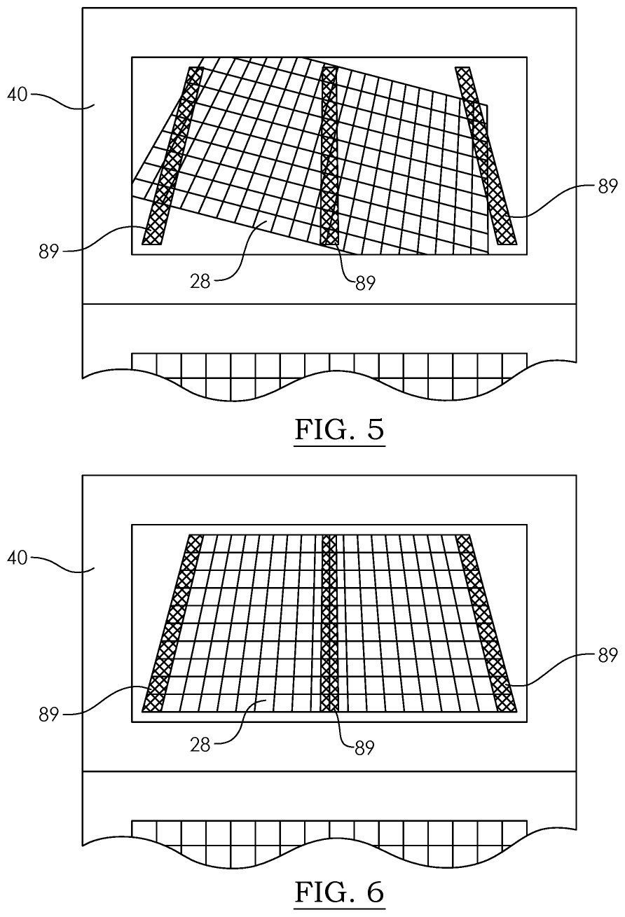

A path 89 image is generated by software and displayed only on the computer (the path is not projected onto the walkway). A walkway 28 image is captured by the camera 36 and displayed on the computer 40. In the event that the camera is not properly aligned with the walkway, the walkway 28 image and the path 89 image will not coincide on screen, as shown in FIG. 5. The walkway 28 image and the path 89 image will coincide when the camera 36 is aligned with the walkway 28, as shown in FIG. 6. Thus, final alignment of the camera is done by viewing the computer screen while adjusting the camera.

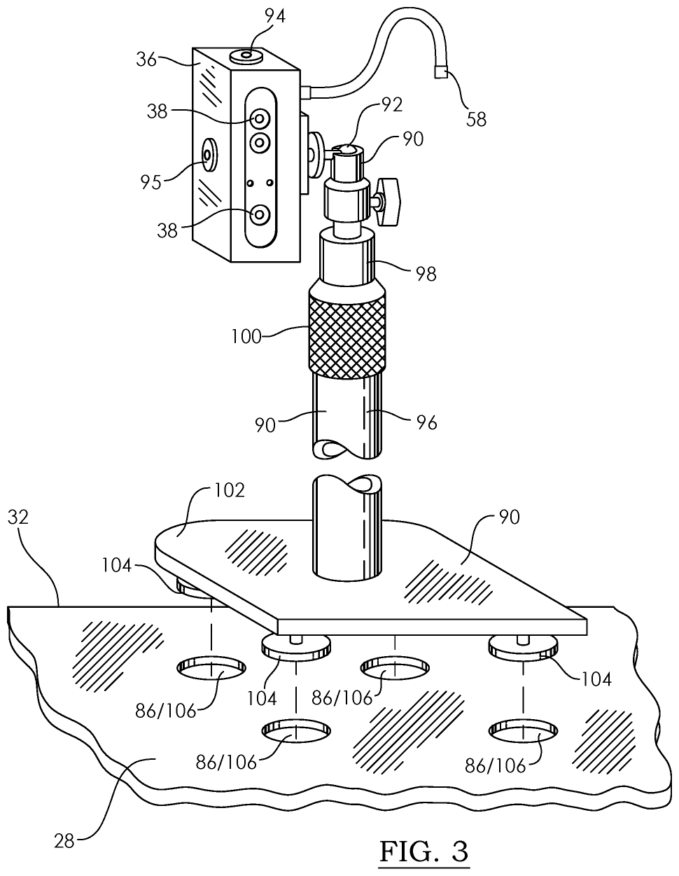

A camera support 90 includes a camera support base 102 juxtaposed with the target 86, as shown in FIG. 3. A first upright element 96 extends upward from the base 102. A second upright element 98 telescopes upward from the first upright element 96. A locking element 100 connects the first and second upright elements. The locking element 100 is adapted for selectively locking and releasing the first 96 and second 98 upright elements to one another. In this manner, the height of the camera 36 above the walkway 28 is selectively adjusted.

A camera mount 92 is attached to the second upright element 98. The camera mount 92 is releasably attached to the camera 36, and is adjustable so as to level the camera. At least one bubble level 94, 95 is mounted on either one of the camera or the camera mount. Preferably, two bubble levels are mounted, one horizontal level 95 on the camera top, and one vertical level 94 on the end. Thus, the camera can be leveled with the lenses disposed transversely or vertically. Typically, the lenses are disposed vertically.

In the preferred embodiment, the walkway target 86 has a plurality of pockets 106 disposed on an upper surface of the walkway 28. The pockets 106 are spaced apart in a predetermined pattern. The camera support base 102 has a plurality of feet 104 that are configured to be received in the pockets 106. The predetermined pattern allows the camera support to be disposed repeatably in only one position, that is, facing toward the first end 30 and centered on the walkway axis 29. The camera support base 102 cannot be turned facing backward or sideways, as the feet 104 will not enter the pockets 106. Hence, the camera will quickly and consistently be positioned in proper alignment with the walkway.

A walkway power means 42 supplies electrical power, and is operatively electrically connected to the walkway 28. The walkway power means 42 includes a power supply 84 plugged into grid power 85.

A camera power means 44 supplies electrical power, and is operatively electrically connected to the camera 36. The camera power means 44 includes the power supply 84 plugged into grid power 85.

A computer power means 46 supplies electrical power, and is operatively electrically connected to the computer 40. The computer power means 46 includes the power supply 84 plugged into grid power 85.

As described above, the walkway power means 42, the camera power means 44, and the computer power means 46, each includes the power supply 84 plugged into grid power 85, which defines a system power means 72. The system power means 72 supplies electrical power through a single, unitary component, the electrical interface module 74, which is connected through connector cables 58 to the walkway 28, to the 3D camera 36, and to the computer 40.

A data means 48 conveys data, and is operatively digitally connected to the walkway 28, the camera 36, and the computer 40. The data means 48 comprises a digital interface module 49 connected through connector cables 58 to the walkway 28, to the 3D camera 36, and to the computer 40. Thus, the data from the walkway 28 and the data from the camera 36 are captured simultaneously.

As described above, the system power means 72 and the data means 48 are separate means for the electrical power and the data signal. However, in the preferred embodiment, the system power means 72 and the data means 48 further comprise a single, unitary interface module 76. The unitary interface module 76 operatively electrically connects electrical conductors, connector cables 58 to the walkway 28, the camera 36, and the computer 40, and to. At the same time, and with the same conductors, the unitary interface module 76 operatively digitally connects digital conductors, connector cables 58 to the walkway 28, the camera 36, and the computer 40. Thus, the biokinetic analysis system 20 conveys both power and data simultaneously.

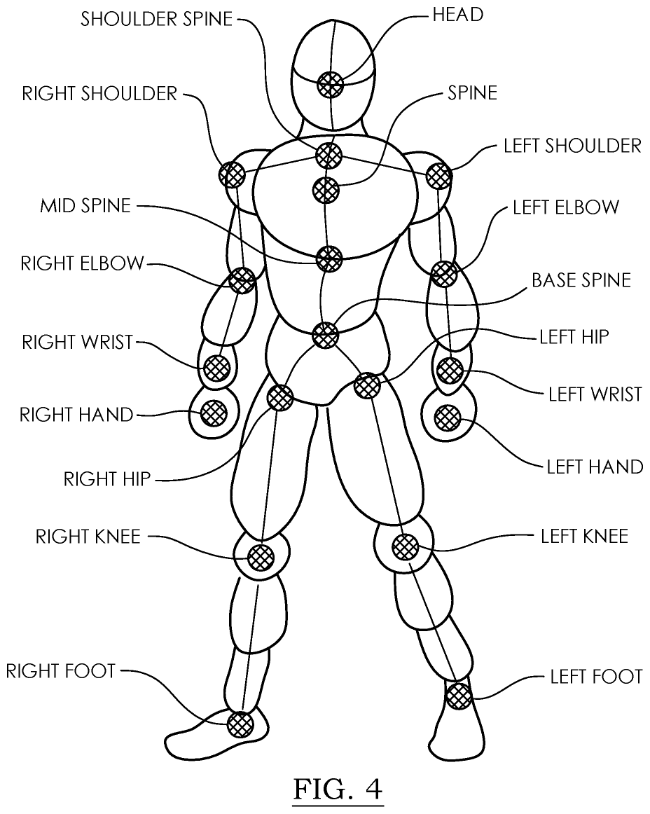

FIG. 4 illustrates the movement of the subject body 24 on the walkway 28 as subject body data, represented graphically on the computer screen. Various portions of the subject body 24 are indicated as circles connected by lines.

The problem with using a depth camera alone for gait analysis is an inadequacy in capturing the motion of the feet. The present invention resolves that problem by using the walkway data, which determines the actual location of each foot on the ground, along with the pressure distribution under each foot. The walkway data and the camera data are captured in real time and integrated into the body analysis process. Thus, the accuracy and precision of the analysis is greatly improved over the prior art.

When data capture of the feet and data capture of the body is synchronized in real time, and integrated digitally, the problem of finding an absolute relationship between the body movement and the foot movement is overcome by the present invention. Now that the problem is solved by the present invention as described and claimed herein, the present invention will provide the clinician with a simple to operate apparatus. The output report is an accurate, easy to understand measurement format in numbers that can be compared over time to see the effect of the treatment.

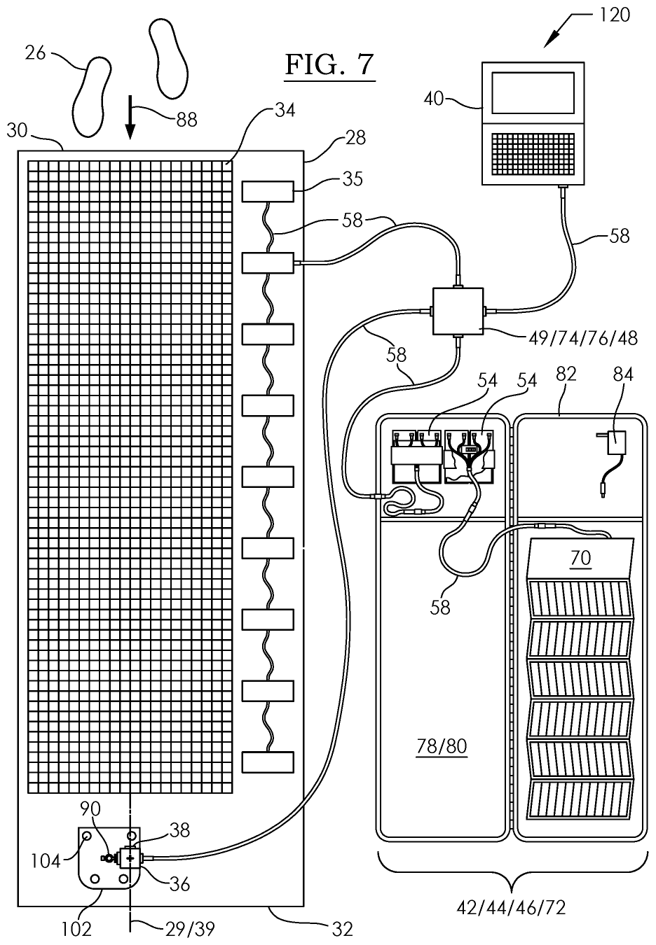

Turning now to FIG. 7, as well as FIGS. 1-6, another biokinetic analysis system 120 is used in connection with a human subject 22 having a subject body 24 and subject feet 26. The biokinetic analysis system 120 is similar to biokinetic analysis system 20 described above, in that the biokinetic analysis system 120 comprises an instrumented walkway 28 extending from a first end 30 to an opposite second end 32. The walkway has a target 86 disposed on the walkway adjacent the second end of the walkway. The walkway 28 has an array of sensors 34 disposed on the walkway 28 so as to detect pressure of the subject feet 26 walking upon the walkway 28. The pressure is collected as subject feet data, conveyed digitally. The walkway has a walkway axis 29 centered transversely on the array of sensors, as described above.

A 3D camera 36 is disposed above the target adjacent the second end 32 of the walkway 28. The camera 36 has two lenses 38 aimed longitudinally to the walkway 28 and toward the first end 30 of the walkway 28. The lenses each have a centerline lens axis 39. Each lens axis 39 and the walkway axis 29 lie generally within a plane that is generally vertical.

A computer 40 is operatively digitally connected to the walkway 28 and to the camera 36. The computer 40 receives the subject feet data from the walkway 28 and the subject body data from the camera 36 simultaneously. The computer 40 will capture the data from the walkway 28 and the camera 36 simultaneously as the subject walks from the walkway first end 30 toward the second end 32.

A path image is generated by software and displayed on the computer 40. A walkway image is captured by the camera and displayed on the computer. In the event that the camera is not properly aligned with the walkway, the walkway image and the path image will not coincide on screen, as shown in FIG. 5. The walkway image and the path image will coincide when the camera is aligned with the walkway, as shown in FIG. 6. Thus, final alignment of the camera is done by viewing the computer screen while adjusting the camera.

A camera support 90 includes a camera support base 102 juxtaposed with the target 86. A first upright element 96 extends upward from the base 102. A second upright element 98 telescopes upward from the first upright element 96. A locking element 100 connects the first and second upright elements. The locking element 100 is adapted for selectively locking and releasing the first and second upright elements to one another. In this manner, the height of the camera 36 above the walkway 28 is selectively adjusted.

A camera mount 92 is attached to the second upright element 98. The camera mount 92 is releasably attached to the camera 36, and is adjustable so as to level the camera. At least one bubble level is mounted on either one of the camera or the camera mount. Preferably, two bubble levels 94, 95 are mounted, one on the camera top and one on the end. Thus, the camera can be leveled with the lenses disposed transversely or preferably vertically.

The walkway target 86 has a plurality of pockets 106 disposed on an upper surface of the walkway 28. The pockets 106 are spaced apart in a predetermined pattern. The camera support base 102 has a plurality of feet 104 that are configured to be received in the pockets 106. The predetermined pattern allows the camera support to be disposed repeatably in only one position, that is, facing toward the first end and centered on the walkway axis.

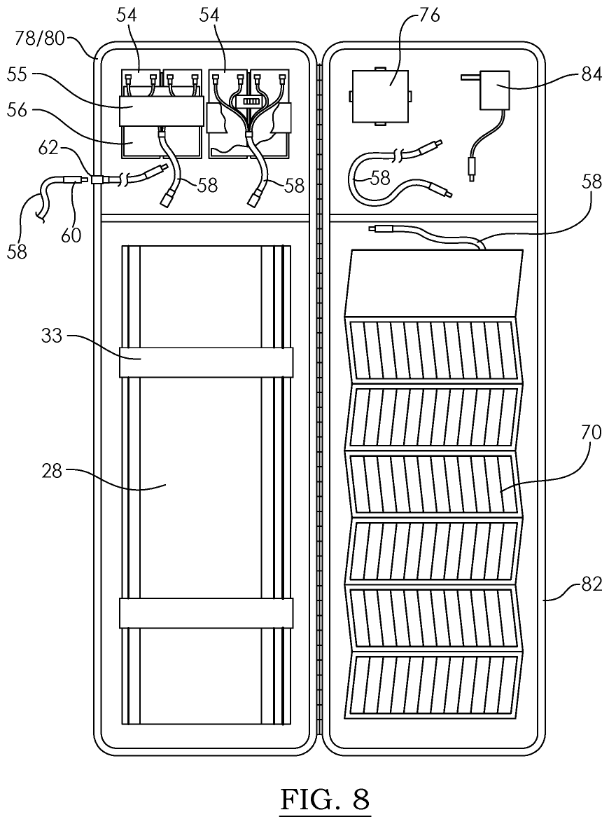

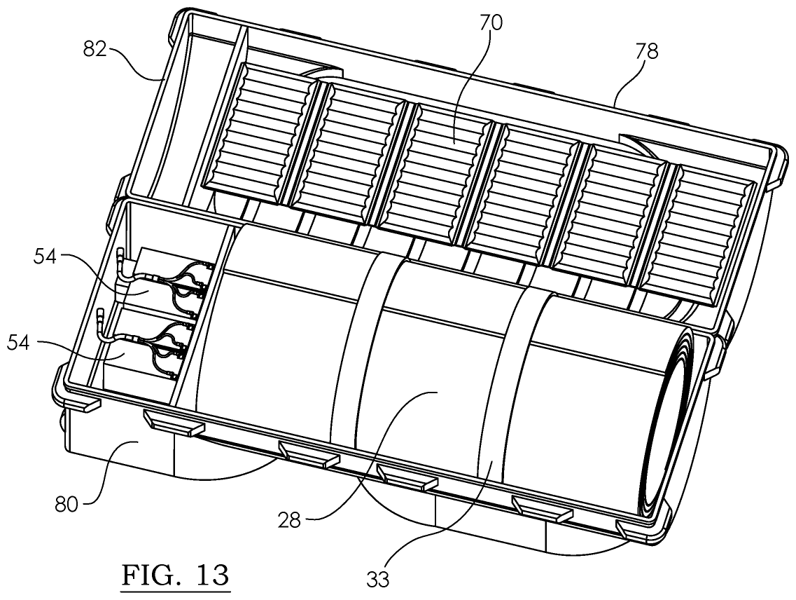

The biokinetic analysis system 120 differs from biokinetic analysis system 20 described above, in that a walkway power means 42 supplies electrical power, and is operatively electrically connected to the walkway 28. In FIGS. 7 and 8, the walkway power means will typically be either an electrical storage battery 50, 52, or a photovoltaic module 70 adapted for receiving solar light and converting the light into electrical power, shown in FIGS. 7, 8, 13, and 15. In one mode, the battery 50, 52, will operate the walkway 28, while the photovoltaic module 70 charges a spare battery. In another mode, the photovoltaic module 70 will operate the walkway 28, while the battery 50, 52, is being charged by a power supply 84 plugged into grid power 85 where available.

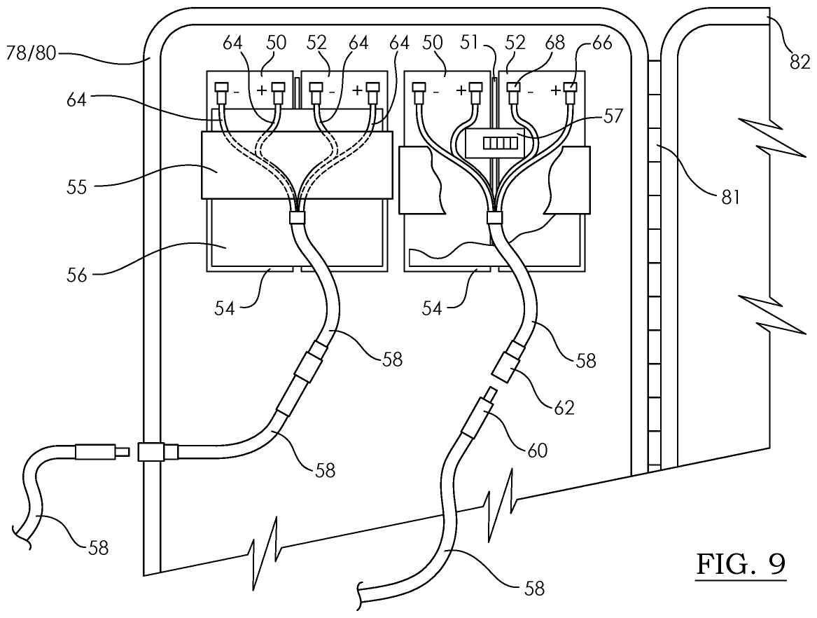

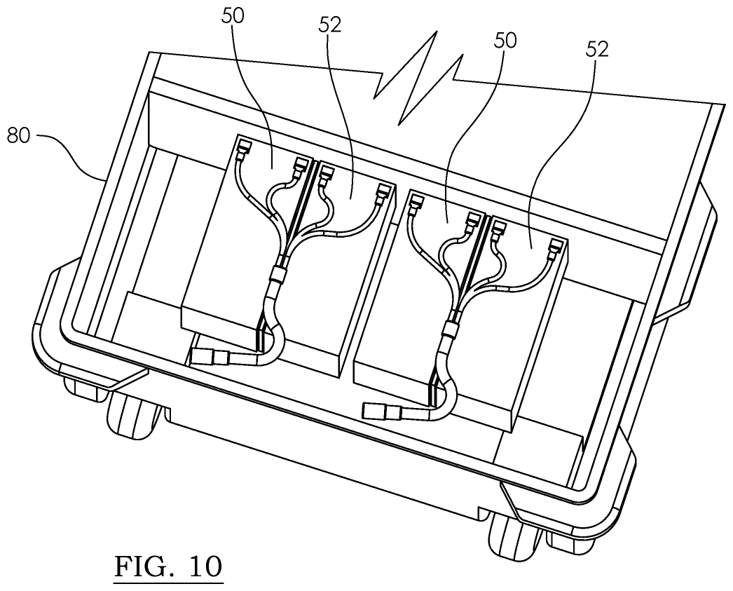

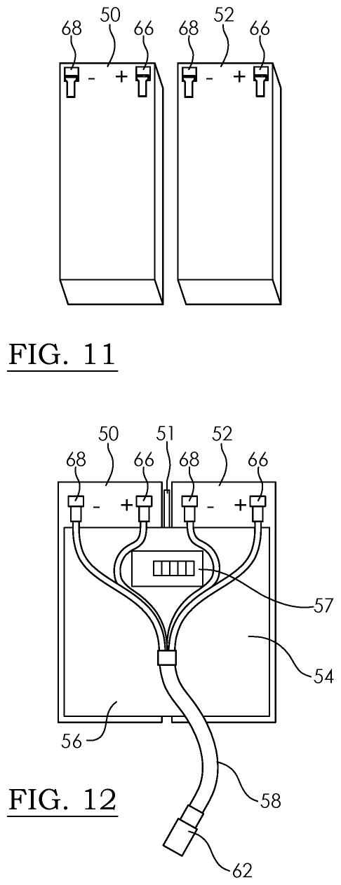

The electrical storage battery has two battery packs 54, 56, each having two batteries 50, 52, as shown in FIGS. 9-12. The batteries 50, 52 are wired together in parallel with connector cables 58 having a male plug 60 on one end and a female socket 62 on the opposite end. Individual wires 64 are connected to the positive 66 and negative 68 terminals of the battery 50, 52. The two batteries 50, 52 are separated by a separation panel 51, which cushions the batteries. The two batteries 50, 52 are covered by a cover panel 53 that cushions and electrically insulates the batteries 50, 52. The two battery packs 54, 56, each are secured by a battery strap 55 against movement. An optional battery charge meter 57 shows the level of power left in the battery pack 54, 56.

A camera power means 44 supplies electrical power, and is operatively electrically connected to the camera 36. The camera power means 44 will typically be either an electrical storage battery 50, 52, or a photovoltaic module 70 adapted for receiving solar light and converting the light into electrical power.

A computer power means 46 supplies electrical power, and is operatively electrically connected to the computer 40. The computer power means 46 will typically be either an electrical storage battery 50, 52, or a photovoltaic module 70 adapted for receiving solar light and converting the light into electrical power.

As described above, the walkway power means 42, the camera power means 44, and the computer power means 46, can be separate power sources. Typically, a system power means 72 is provided for supplying electrical power in a single, unitary component, the electrical interface module 74. The system power means 72 will typically be either an electrical storage battery 50, 52, or a photovoltaic module 70 adapted for receiving solar light and converting the light into electrical power. The system power means 72 is operatively electrically connected to the walkway 28, the camera 36, and the computer 40.

As described above, the system power means 72 and the data means 48 are separate means for the electrical power and the data signal. However, the system power means 72 and the data means 48 preferably comprise a single, unitary interface module 76. The unitary interface module 76 operatively electrically connects connector cables 58 (electrical conductors) to the walkway 28, the camera 36, and the computer 40, and to either the battery 50, 52 or the photovoltaic module 70. At the same time, and with the same conductors, the unitary interface module 76 operatively digitally connects connector cables 58 (digital conductors) to the walkway 28, the camera 36, and the computer 40. Thus, the biokinetic analysis system 20 conveys both power and data simultaneously, and over the same conductors.

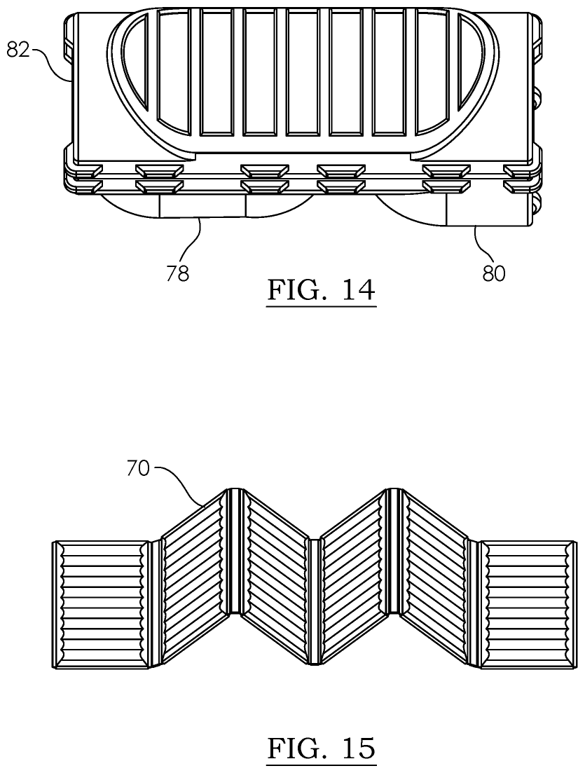

In FIGS. 8, 13, and 14, a transport case 78 is provided for receiving and transporting the walkway 28, the camera 36, the computer 40, the battery 54, 56, the photovoltaic module 70, and the unitary interface module 76. The transport case 78 has a lower portion 80 and an upper portion, or lid 82. The transport case lower portion 80 and upper portion 82 are connected by a transport case hinge 81. The walkway 28 is rolled up and secured in the transport case 78 by walkway straps 33. The transport case 78 can be a commonly available golf case.

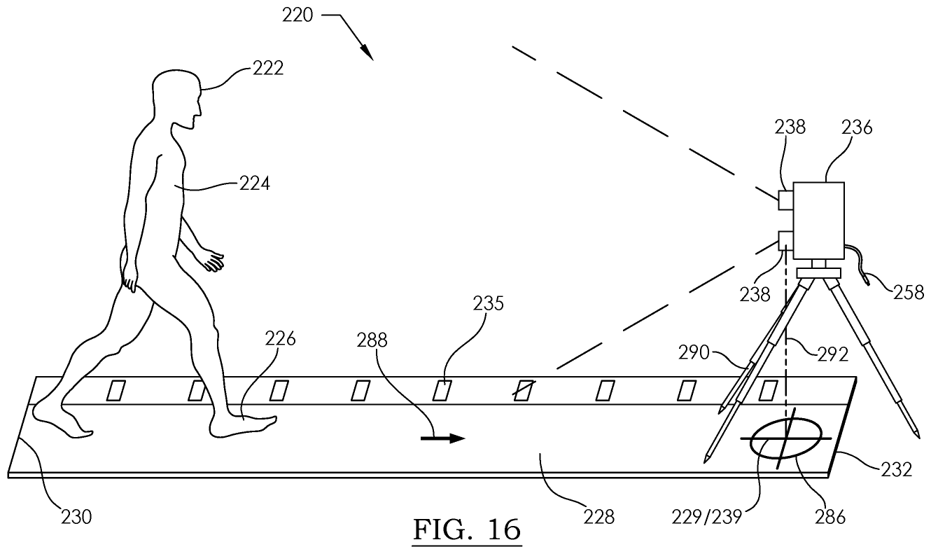

Referring now to FIGS. 16-18, yet another biokinetic analysis system 220 is used in connection with a human subject 222 having a subject body 224 and subject feet 226. The biokinetic analysis system 220 is similar to biokinetic analysis system 20 described above, in that system 220 comprises an instrumented walkway 228 extending from a first end 230 to an opposite second end 232. The walkway has a target 286 disposed on the walkway adjacent the second end 232 of the walkway. The walkway 228 has an array of sensors 234 disposed on the walkway 228 so as to detect pressure of the subject feet 226 walking upon the walkway 228. The pressure is collected as subject feet data, conveyed digitally. The walkway has a walkway axis 229 centered transversely on the array of sensors 234.

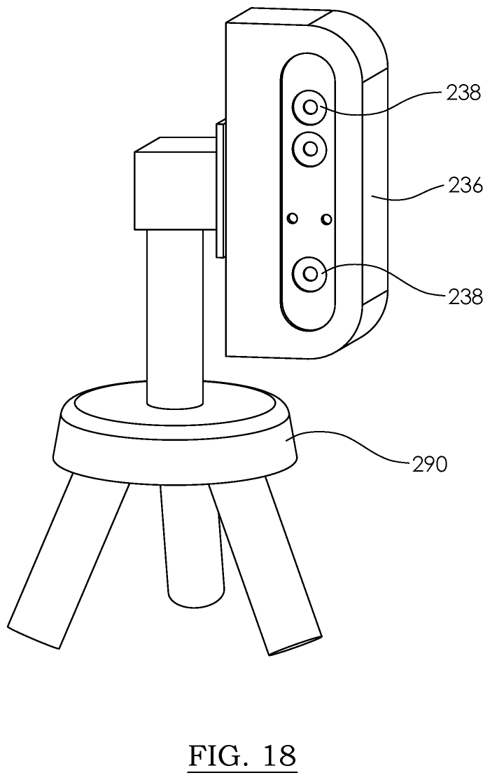

A 3D camera 236 is disposed above the target 286 adjacent the second end 232 of the walkway 228. The camera 236 has two lenses 238 aimed longitudinally to the walkway 228 and toward the first end 230 of the walkway 228. The lenses each have a centerline lens axis 239. Each lens axis 239 and the walkway axis 229 lie generally within a plane that is generally vertical. The 3D camera 236 is disposed with the lenses 238 spaced apart one above the other, as shown in FIGS. 16 and 18.

A computer 240 is operatively digitally connected to the walkway 228 and to the camera 236. The computer 240 receives the subject feet data from the walkway 228 and the subject body data from the camera 236 simultaneously. The computer 240 will capture the data from the walkway 228 and the camera 236 simultaneously as the subject walks from the walkway first end 230 toward the second end 232.

A walkway power means 242 supplies electrical power, and is operatively electrically connected to the walkway 228. The walkway power means 242 includes a power supply 284 plugged into grid power 285.

A camera power means 244 supplies electrical power, and is operatively electrically connected to the camera 236. The camera power means 244 includes the power supply 84 plugged into grid power 285.

A computer power means 246 supplies electrical power, and is operatively electrically connected to the computer 240. The computer power means 246 includes the power supply 284 plugged into grid power 285.

As described above, the walkway power means 242, the camera power means 244, and the computer power means 246, each includes the power supply 284 plugged into grid power 285, which defines a system power means 272. The system power means 272 supplies electrical power through a single, unitary component, the electrical interface module 274, which is connected through connector cables 258 to the walkway 228, to the 3D camera 236, and to the computer 240.

A data means 248 conveys data, and is operatively digitally connected to the walkway 228, the camera 236, and the computer 240. The data means 248 comprises a digital interface module 249 connected through connector cables 258 to the walkway 228, to the 3D camera 236, and to the computer 240. Thus, the data from the walkway 228 and the data from the camera 236 are captured simultaneously.

As described above, the system power means 272 and the data means 248 are separate means for the electrical power and the data signal. Preferably, the system power means 272 and the data means 248 further comprise a single, unitary interface module 276. The unitary interface module 276 operatively electrically connects connector cables 258 (power conductors) to the walkway 228, the camera 236, the computer 240, and to the power supply 284. At the same time, and with the same conductors, the unitary interface module 276 operatively digitally connects connector cables 258 (digital conductors) to the walkway 228, the camera 236, and the computer 240. Thus, the biokinetic analysis system 220 conveys both power and data simultaneously, and over the same conductors.

The biokinetic analysis system 220 differs from biokinetic analysis system 20 described above, in that the 3D camera 236 is mounted on a camera support 290, which may be a tripod with an articulated head, but any sturdy support will serve. The tripod head or other suitable support will allow the 3D camera 236 to be rotated from the commonly seen prior art position with the lenses 238 spaced apart transversely, to a position with the lenses displaced vertically. The preferred placement has the lenses 238 positioned one above the other, because the aspect ratio of the subject body 224 is greater in height than it is in width. The human subject 222 is walking toward the camera, hence a wide angle of field is not needed. Nor is the camera panned sideways, but is fixed in position. The height of the 3D camera 236 is selectively adjusted to be at approximately half the height of the subject 222.

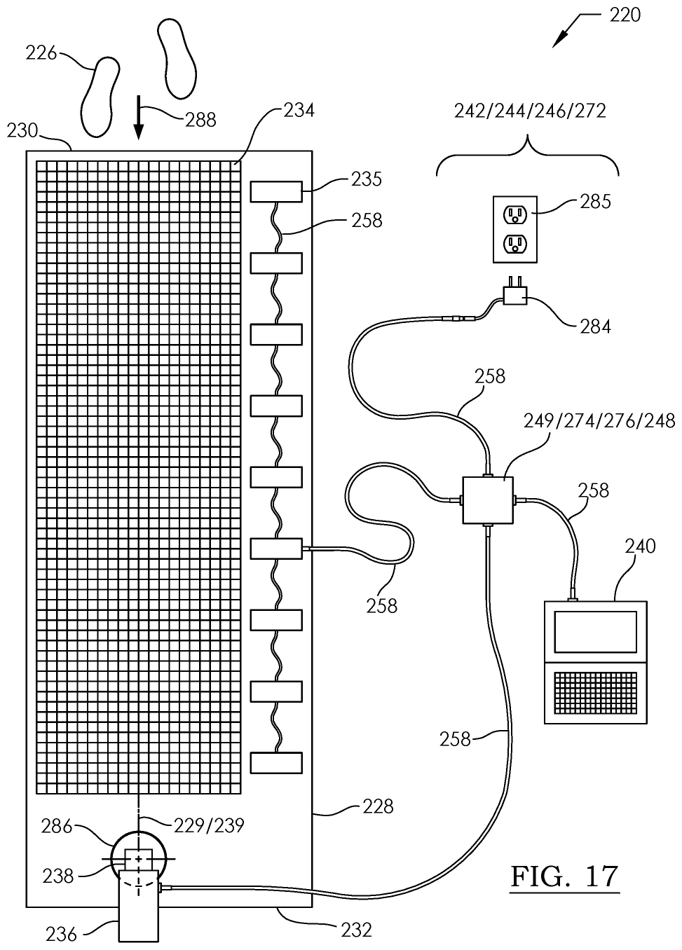

The 3D camera 236 is disposed above the target 286, and is aligned with the target. The camera lens 238 is shown positioned over the target 286, in FIGS. 16 and 17. Any element of the camera can be used as a reference, provided the same element is always used, for consistent results. A plumb line 292 (virtual line) shows the positioning of the camera over the target 286. The camera 236 can be visually positioned over the target 286. A plumb bob (not shown), or a level (not shown) or other aid can optionally be used for positioning.

It will be appreciated that variants of the above-disclosed and other features and functions, or alternatives thereof, may be desirably combined into many other different systems or applications. Various presently unforeseen or unanticipated alternatives, modifications, variations, or improvements therein may be subsequently made by those skilled in the art which are also intended to be encompassed by the following claims.

PARTS LIST

| PART | |
|---|---|
| NO. | DESCRIPTION |
| 20 | biokinetic analysis system |
| 22 | human subject |
| 24 | subject body |
| 26 | subject feet |
| 28 | instrumented walkway |
| 29 | centerline walkway axis |
| 30 | walkway first end |
| 32 | walkway second end |
| 33 | walkway straps |
| 34 | sensors |
| 35 | circuit boards |
| 36 | 3D camera |
| 38 | lenses |
| 39 | centerline lens axis |
| 40 | computer |
| 42 | walkway power means |
| 44 | camera power means |
| 46 | computer power means |
| 48 | data means |
| 49 | digital interface module |
| 50 | electrical storage battery 1 |
| 51 | separation panel |
| 52 | electrical storage battery 2 |
| 54 | battery pack |
| 55 | battery strap |
| 56 | cover panel |
| 57 | battery charge meter |
| 58 | connector cables |
| 60 | male plug |
| 62 | female socket |
| 64 | wires |
| 66 | positive terminal |
| 68 | negative terminal |
| 70 | photovoltaic module |
| 72 | system power means |
| 74 | electrical interface module |
| 76 | unitary interface module |
| 78 | transport case |
| 80 | transport case lower |
| 81 | transport case hinge |
| 82 | transport case upper |
| 84 | power supply |
| 85 | grid power |
| 86 | target |
| 88 | walking direction |
| 89 | path |
| 90 | camera support |
| 92 | camera mount |
| 94 | vertical level |
| 95 | horizontal level |
| 96 | first upright element |
| 98 | second upright element |
| 100 | locking element |
| 102 | camera support base |
| 104 | camera support feet |
| 106 | camera locating pockets |
| 120 | biokinetic analysis system |
| 220 | biokinetic analysis system |
| 222 | human subject |
| 224 | subject body |
| 226 | subject feet |
| 228 | instrumented walkway |
| 229 | centerline walkway axis |
| 230 | walkway first end |
| 232 | walkway second end |
| 234 | sensors |
| 235 | circuit boards |
| 236 | 3D camera |
| 238 | lenses |
| 239 | center axis lenses |
| 240 | computer |
| 242 | walkway power means |
| 244 | camera power means |
| 246 | computer power means |
| 248 | data means |
| 249 | digital interface module |
| 258 | connector cables |
| 272 | system power means |
| 274 | electrical interface module |
| 276 | unitary interface module |
| 284 | power supply |
| 285 | grid power |
| 286 | target |
| 288 | walking direction |
| 290 | camera support |
| 292 | plumb line |

What is claimed is:

1. A biokinetic analysis system, for use in connection with a human subject having a subject body and subject feet, the biokinetic analysis system comprising:
an instrumented walkway extending from a first end to an opposite second end, the walkway being adapted for lying upon a floor, the walkway having an array of sensors disposed on the walkway so as to detect pressure of the subject feet walking upon the walkway as subject feet data, the walkway being adapted to convey the subject feet data digitally;
a 3D camera disposed adjacent the walkway, the camera having two lenses aimed toward the walkway, the camera being adapted to optically capture movement of the subject body on the walkway as subject body data, the camera being adapted to convey the subject body data digitally;
a computer operatively digitally connected to the walkway and to the camera, the computer being adapted to receive the subject feet data from the walkway and the subject body data from the camera simultaneously, the computer being programmed to analyze and store the data;
walkway power means for supplying electrical power, the walkway power means being operatively electrically connected to the walkway;
camera power means for supplying electrical power, the camera power means being operatively electrically connected to the camera;
computer power means for supplying electrical power, the computer power means being operatively electrically connected to the computer; and
data means for conveying data, the data means being operatively digitally connected to the walkway, the camera, and the computer; wherein
the subject will walk from the walkway first end toward the walkway second end; and
the data from the walkway and the data from the camera are captured simultaneously.

2. The biokinetic analysis system of claim 1, wherein:
the walkway includes a centerline walkway axis extending between the first and second ends and centered transversely on the array of sensors;
the camera lenses each include a centerline lens axis;
the 3D camera is disposed above the walkway adjacent the second end of the walkway, with the camera lenses being aimed longitudinally to the walkway toward the first end of the walkway; and
the 3D camera is disposed with the lenses spaced apart one above the other, and with each lens axis aligned generally parallel to the walkway axis.

3. The biokinetic analysis system of claim 2, wherein:
the walkway has a target disposed on the walkway adjacent the second end of the walkway; and
the 3D camera is disposed above the target, so as to align the lenses with the walkway; wherein
the lens axis and the walkway axis lie generally within a plane that is generally vertical.

4. The biokinetic analysis system of claim 3, further comprising a camera support, including:
a camera support base juxtaposed with the target; and
an upright element extending upward from the base; wherein
the camera is supported by the upright element.

5. The biokinetic analysis system of claim 4, wherein the camera support further comprises:
a first upright element extending upward from the base;
a second upright element telescoping upward from the first upright element;
a locking element connecting the first and second upright elements, the locking element being adapted for selectively locking and releasing the first and second upright elements to one another, so as to selectively adjust the height of the camera above the walkway; and
a camera mount attached to the second upright element, the camera mount being adapted to be releasably attached to the camera, the camera mount being adjustable so as to level the camera.

6. The biokinetic analysis system of claim 5, wherein:
the walkway target further comprises a plurality of pockets disposed on an upper surface of the walkway, the pockets being spaced apart in a predetermined pattern; and
camera support base further comprises a plurality of feet adapted for being received in the pockets; whereby
the predetermined pattern allows the camera support to be disposed repeatably in only one position, so as to align the camera with the walkway.

7. The biokinetic analysis system of claim 2, further comprising:
a path image generated by software and displayed on the computer; and
a walkway image captured by the camera and displayed on the computer; wherein
the walkway image and the path image will not coincide when the camera is misaligned with the walkway; and
the walkway image and the path image will coincide when the camera is aligned with the walkway.

8. The biokinetic analysis system of claim 1, wherein the walkway power means is selected from the group consisting of:
at least one electrical storage battery;
a photovoltaic module adapted for receiving solar light and converting the light into electrical power; and
a power supply operatively electrically connected to utility grid power.

9. The biokinetic analysis system of claim 1, wherein the camera power means is selected from the group consisting of:
at least one electrical storage battery;
a photovoltaic module adapted for receiving solar light and converting the light into electrical power; and
a power supply operatively electrically connected to utility grid power.

10. The biokinetic analysis system of claim 1, wherein the computer power means is selected from the group consisting of:
at least one electrical storage battery;
a photovoltaic module adapted for receiving solar light and converting the light into electrical power; and
a power supply operatively electrically connected to utility grid power.

11. The biokinetic analysis system of claim 1, further comprising a system power means for supplying electrical power, the system power means being operatively electrically connected to the walkway, the camera, and the computer, the system power means being selected from the group consisting of:
at least one electrical storage battery;
a photovoltaic module adapted for receiving solar light and converting the light into electrical power; and
a power supply operatively electrically connected to utility grid power.

12. The biokinetic analysis system of claim 11, wherein the system power means further comprises an electrical interface module for operatively electrically connecting electrical conductors to the walkway, the camera, and the computer.

13. The biokinetic analysis system of claim 1, wherein the data means further comprises a digital interface module operatively digitally connected to the walkway, the camera, and the computer.

14. The biokinetic analysis system of claim 11, wherein the system power means and the data means further comprise a unitary interface module for operatively electrically connecting the walkway, the camera, and the computer, and for operatively digitally connecting the walkway, the camera, and the computer, thereby conveying both power and data simultaneously.

15. The biokinetic analysis system of claim 1, further comprising a transport case for receiving and transporting the biokinetic analysis system.

16. A biokinetic analysis system, for use in connection with a human subject having a subject body and subject feet, the biokinetic analysis system comprising:
an instrumented walkway extending from a first end to an opposite second end, the walkway being adapted for lying upon a floor, the walkway having a target disposed on the walkway adjacent the second end of the walkway, the walkway having an array of sensors disposed on the walkway so as to detect pressure of the subject feet walking upon the walkway as subject feet data, the walkway being adapted to convey the subject feet data digitally;
a 3D camera disposed above the target adjacent the second end of the walkway and spaced apart from the array of sensors, the camera having two lenses aimed longitudinally to the walkway and toward the first end of the walkway, the camera being adapted to optically capture movement toward the camera of the subject body on the walkway as subject body data, the camera being adapted to convey the subject body data digitally;
a computer operatively digitally connected to the walkway and to the camera, the computer being adapted to receive the subject feet data from the walkway and the subject body data from the camera simultaneously, the computer being programmed to analyze and store the data;
a system power means for supplying electrical power, the system power means being operatively electrically connected to the walkway, the camera, and the computer, wherein the system power means is selected from the group consisting of:
at least one electrical storage battery;
a photovoltaic module adapted for receiving solar light and converting the light into electrical power; and
a power supply operatively electrically connected to utility grid power; and
data means for conveying data, the data means being operatively digitally connected to the walkway, the camera, and the computer; wherein
the subject will walk from the walkway first end toward the walkway second end; and
the data from the walkway and the data from the camera are captured simultaneously.

17. The biokinetic analysis system of claim 16, wherein the system power means and the data means further comprise a unitary interface module for operatively electrically connecting the walkway, the camera, and the computer, and for operatively digitally connecting the walkway, the camera, and the computer, thereby conveying both power and data simultaneously.

18. The biokinetic analysis system of claim 16, wherein:
the walkway includes a centerline walkway axis extending between the first and second ends and centered transversely on the array of sensors; and
the 3D camera lenses each include a centerline lens axis and are disposed with the lenses spaced apart one above the other; wherein
each lens axis and the walkway axis lie generally within a plane that is generally vertical.

19. The biokinetic analysis system of claim 18, further comprising a camera support, including:
a camera support base juxtaposed with the target;
a first upright element extending upward from the base;
a second upright element telescoping upward from the first upright element;
a locking element connecting the first and second upright elements, the locking element being adapted for selectively locking and releasing the first and second upright elements to one another, so as to selectively adjust the height of the camera above the walkway;
a camera mount attached to the second upright element, the camera mount being adapted to be releasably attached to the camera, the camera mount being adjustable so as to level the camera; and
at least one bubble level mounted on a one of the camera and the camera mount.

20. The biokinetic analysis system of claim 19, wherein:
the walkway target further comprises a plurality of pockets disposed on an upper surface of the walkway, the pockets being spaced apart in a predetermined pattern; and
camera support base further comprises a plurality of feet adapted for being received in the pockets; whereby
the predetermined pattern allows the camera support to be disposed repeatably in only one position, so as to align the camera with the walkway.

21. The biokinetic analysis system of claim 16, further comprising:
a path image generated by software and displayed on the computer; and
a walkway image captured by the camera and displayed on the computer; wherein
the walkway image and the path image will not coincide when the camera is misaligned with the walkway; and
the walkway image and the path image will coincide when the camera is aligned with the walkway.

22. A biokinetic analysis system, for use in connection with a human subject having a subject body and subject feet, the biokinetic analysis system comprising:
an instrumented walkway extending from a first end to an opposite second end, the walkway being adapted for lying upon a floor, the walkway having a target disposed on the walkway adjacent the second end of the walkway, the walkway having an array of sensors disposed on the walkway so as to detect pressure of the subject feet walking upon the walkway as subject feet data, the walkway being adapted to convey the subject feet data digitally, the walkway having a centerline walkway axis extending between the first and second ends and centered transversely on the array of sensors;
a 3D camera disposed above the target adjacent the second end of the walkway and spaced apart from the array of sensors, the camera having two lenses aimed longitudinally to the walkway and toward the first end of the walkway, the lenses each having a centerline lens axis and being disposed with the lenses spaced apart one above the other, wherein each lens axis and the walkway axis lie generally within a plane that is generally vertical, the camera being adapted to optically capture movement toward the camera of the subject body on the walkway as subject body data, the camera being adapted to convey the subject body data digitally;
a computer operatively digitally connected to the walkway and to the camera, the computer being adapted to receive the subject feet data from the walkway and the subject body data from the camera simultaneously, the computer being programmed to analyze and store the data, wherein the subject will walk from the walkway first end toward the walkway second end, and the data from the walkway and the data from the camera will be captured simultaneously;
a unitary interface module including:
a system power means for operatively electrically connecting the walkway, the camera, and the computer; and
a data means for operatively digitally connecting the walkway, the camera, and the computer, thereby conveying both power and data simultaneously;
a path image generated by software and displayed on the computer; and
a walkway image captured by the camera and displayed on the computer; wherein
the walkway image and the path image will not coincide when the camera is misaligned with the walkway; and
the walkway image and the path image will coincide when the camera is aligned with the walkway.

23. The biokinetic analysis system of claim 22, wherein the system power means is selected from the group consisting of:
at least one electrical storage battery;
a photovoltaic module adapted for receiving solar light and converting the light into electrical power; and
a power supply operatively electrically connected to utility grid power.

24. The biokinetic analysis system of claim 22, further comprising a camera support, including:
a camera support base juxtaposed with the target;
a first upright element extending upward from the base;
a second upright element telescoping upward from the first upright element;
a locking element connecting the first and second upright elements, the locking element being adapted for selectively locking and releasing the first and second upright elements to one another, so as to selectively adjust the height of the camera above the walkway;
a camera mount attached to the second upright element, the camera mount being adapted to be releasably attached to the camera, the camera mount being adjustable so as to level the camera; and
at least one bubble level mounted on a one of the camera and the camera mount.

25. The biokinetic analysis system of claim 24, wherein:
the walkway target further comprises a plurality of pockets disposed on an upper surface of the walkway, the pockets being spaced apart in a predetermined pattern; and
camera support base further comprises a plurality of feet adapted for being received in the pockets; whereby
the predetermined pattern allows the camera support to be disposed repeatably in only one position, so as to align the camera with the walkway.
